(19) United States
(12) Patent Application Publication
(10) Pub. No.: US 2016/0244810 A1
(43) Pub. Date: Aug. 25, 2016

(54) METHOD FOR STEADYING THERMAL CONVECTION FLOW FIELD IN SOLUTION DURING THERMAL CONVECTIVE POLYMERASE CHAIN REACTION

(71) Applicant: GeneReach Biotechnology Corp., Taichung (TW)

(72) Inventors: Chen SU, Taichung (TW); Ping-Hua TENG, Taichung (TW); Chien-Chung JENG, Taichung (TW)

(21) Appl. No.: 15/143,643

(22) Filed: May 2, 2016

Related U.S. Application Data
(63) Continuation-in-part of application No. 14/383,905, filed on Oct. 31, 2014.

(51) Int. Cl. C12Q 1/68; C12P 19/34

(52) U.S. Cl. CPC C12Q 1/686; C12P 19/34

(57) ABSTRACT

A method for steadying a thermal convection flow field in a PCR reaction solution during a thermal convective polymerase chain reaction (PCR) includes steps as follows. A PCR tube is provided. A PCR reaction solution is filled in the PCR tube. A bottom of the PCR tube is heated so that a thermal convection is induced. And the PCR to be is tilted at a tilt angle relative to a vertical line over a ground for causing a single-loop flow in the thermal convection flow field in the PCR reaction solution, whereby a denaturation an annealing reaction and an extension reaction occur sequentially and repeatedly in different temperature regions of the PCR reaction solution.

### RELATED APPLICATIONS

This application is a continuation-in-part of U.S. patent application Ser. No. 14/383,905, filed Oct. 31, 2014, which is the US National Phase application of PCT No. PCT/CN2012/072125, filed Mar. 9, 2012, all of which are herein incorporated by reference.

### BACKGROUND

1. Technical Field

The present disclosure relates to a method for enhancing the performance consistency of thermal convection polymerase chain reaction (PCR). M ore particularly the present disclosure relates to a method for steadying thermal convection flow field in e PCR reaction solution during thermal convective PCR.

2. Description of Related Art

For genetic research such as genetics and molecular biology or the detection of animal and plant diseases, it needs to amplify a few copies of nucleic acids by nucleic acid amplification such as polymerase chain reaction (PCR), from a small amount of nucleic acid samples to the amount which can be detected in a short time. The nucleic acid amplification products can be further hybridized to nucleic acid probes conjugated with fluorescent, radioactive substances or calorimetric enzyme, thus producing fluorescence, radiation image or color reaction.

The aforementioned process of the polymerase chain reaction includes three main steps: denaturation annealing reaction and extension reaction, and the required reaction temperatures of the three main steps are different. Nowadays the sample of commercialized PCR amplification technology includes the template DNA for amplification, the oligonucleotide primers which are complementary to particular sequences of each strand of template DNA, thermostable DNA polymerase, and deoxy-ribonucleoside triphosphate (dNTP), then the reaction repeats heating and cooling the sample, and the sample is circulated between different temperatures to amplify the particular part of template DNA nucleic acid sequence.

In the aforementioned denaturation, the sample is heated to high temperatures, causing the double-stranded template DNA to be separated into single-stranded DNA; in the annealing reaction, the sample is coaled to a lower temperature, allowing annealing of the primers to the single-stranded DNA templates which are formed in denaturation to form a DNA-primer hybrid; in the extension reaction, the sample is maintained at an appropriate temperature, and the primer of the DNA-primer hybrid can be extended to synthesize a new DNA strand complementary to each of the DNA template through the action of DNA polymerase; the DNA sequences between the binding sites of the forward primer and the reverse primer can be replicated in each cycle consisting of three main steps.

The temperature of the sample tube of conventional PCR reaction apparatus is controlled by heat conduction, for example, the PCR sample tube is in contact with a solid metal block of high thermal conductivity, changing the temperature of the sample tube by heating and cooling to reach the required temperature of the PCR sample tube. This type of PCR reaction apparatus is called thermal cycling PCR apparatuses. The drawback of the thermal cycling PCR apparatuses is that it need to spend extra time and energy for heating and cooling the material other than the polymerase chain reaction sample. Thus the procedure of the thermal cycling PCR apparatuses is not efficient addition, the thermal cycling PCR apparatuses are usually very expensive due to the precise characteristics of the apparatuses.

Another type of PCR reaction apparatus is called thermal convective PCR apparatuses. The procedure of the thermal convective PCR method is heating the bottom of a container containing the PCR reaction solution and a temperature gradient along the container can be generated in order to perform the thermal convective PCR. Accordingly, the denaturation, the annealing reaction and the extension reaction occur sequentially and repeatedly in different temperature regions of the container established by the thermal convection allowing the PCR to be completed.

The ideal convection flow field in the thermal convective PCR is a single-loop flow from the bottom of the PCR reaction solution to the top of the PCR reaction solution, so that the performance of t he thermal convective PCR is consistent among different PCR tubes under the same reaction condition. However, when the tube is placed in a vertical position, the convection flow field in the thermal convective PCR not only the single-loop flow but also a two-loop flow or a multi-loop flow are formed, due to the difference of the flow rates at the top of the PCR reaction solution and the flow rates at the bottom of the PCR reaction solution due to different corresponding driving force in different temperature regions. Accordingly, the PCR reaction solution only circulates at a certain temperature region and cannot flow through the full temperature regions. Thus the performances of the thermal convective PCR are not consistent in different tubes containing the same PCR reaction solution.

### SUMMARY

According to one aspect of the present disclosure, a method for steadying a thermal convection flow field in a PCR reaction solution during a thermal convective polymerase chain reaction (PCR) includes steps as follows. A PCR tube is provided. A PCR reaction solution is filled in the PCR tube. A bottom of the PCR tube is heated so that a thermal convection is induced. And the PCR tube is tilted at a tilt angle relative to a vertical line over a ground for causing a single-loop flow in the thermal convection flow field in the PCR reaction solution, whereby a denaturation, an annealing reaction and an extension reaction occur sequentially and repeatedly in different temperature regions of the PCR reaction solution.

### BRIEF DESCRIPTION OF THE DRAWINGS

FIG. 1 is a schematic diagram of the thermal convection circulation in the sample tube which is in a vertical position during the biochemical reaction.

FIG. 2 is a schematic diagram of the thermal convection circulation in the sample tube which is in a horizontal position during the biochemical reaction.

FIG. 3 is a schematic diagram of the thermal convection circulation in he sample tube which is in an inclined position during the biochemical reaction.

FIG. 4 is a flow chart of operation of one embodiment of the present disclosure.

FIG. 5 is a schematic diagram of a using stag before adjusting an angle of the tube according to one embodiment of the present disclosure.

FIG. 6 is a schematic diagram of a using state after adjusting an angle of the tube according to one embodiment of the present disclosure.

FIG. 7 is a schematic diagram of an operation of the tube at a tilt angle according to one embodiment of the present disclosure.

FIG. 8 is a flow diagram showing a method for steadying a thermal convection flow field in a PCR reaction solution during a thermal convective polymerase chain reaction (PCR) according to another embodiment of the present disclosure.

FIG. 9 is a cross-sectional view of PCR tube according to another embodiment of the present disclosure.

FIG. 10A is a schematic diagram of an ideal thermal convection flow field in the PCR tube which is in a vertical position during the thermal convective PCR.

FIG. 10B is a schematic diagram of an actual thermal convection flow field in the PCR tube which is in the vertical position during the thermal convective PCR.

FIG. 10C is a schematic diagram of the thermal convection flow field in the PCR tube which is in an inclined position during the thermal convective PCR according to another embodiment of the present disclosure,

FIGS. 11A to 11F are diagrams of computer-simulation fluid dynamics in the thermal convection flow field in the PCR tube at different tilt angles according to one example of the present disclosure.

FIGS. 12A and 12B are diagrams of the fluorescent signals generated in the thermal convective PCR, wherein the PCR tube is in the vertical position during the thermal convective PCR.

FIGS. 13A and 13B are diagrams of the fluorescent signals generated in the thermal convective PCR according to another embodiment of the present disclosure, wherein the PCR tube is tilted at 20 during the thermal convective PCR,

### DETAILED DESCRIPTION

Reference will now be made in detail to the p resent embodiments of the present disclosure, examples of which are illustrated in the accompanying drawings. That must be explained first, the elements of the embodiments are drawn for easily explaining the ratio, size, and degrees of deformation and position shift, and are not drawn to scale. Wherever possible, the same reference numbers are used in the drawings and the description to refer to the same or like parts.

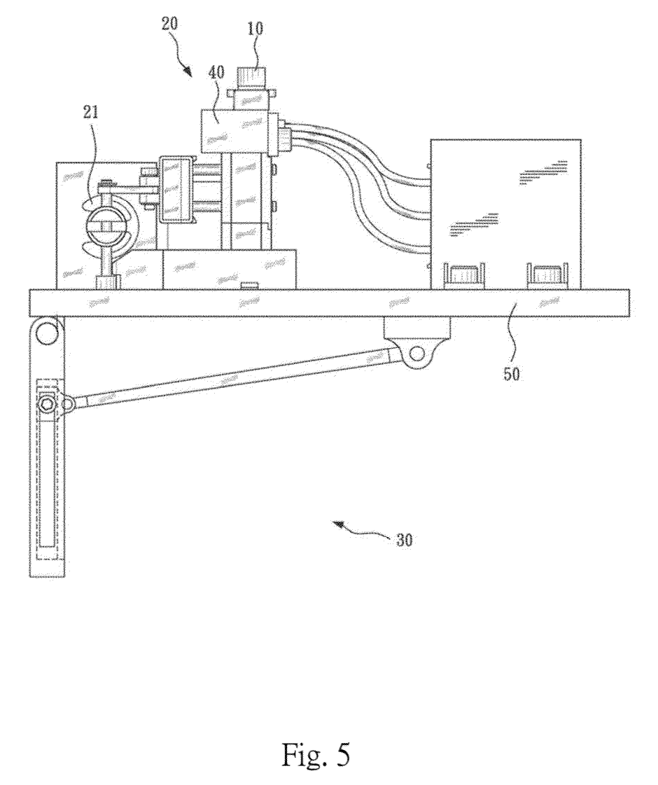

The present disclosure relates to a device for controlling thermal convection velocity of a biochemical reaction and method for the same. That must be explained first, each component and setting in the drawings are only for easily explaining the present disclosure. The present disclosure covers variations of the reaction apparatus and is not limited to the illustration shown in the drawings. In FIG. 5, to achieve the purpose that the present disclosure can control the thermal convection velocity required for the biochemical reaction, the elements related to the main technical contents of the present disclosure include a tube 10, a heating source 20, and a flow rate adjusting apparatus 30.

The tube 10, provided for nucleic acid extraction, is filled with at least one buffer 11 of the biochemical reaction and at least one fluorescent substance. The buffer 11 can be the buffer of polymerase chain reaction (PCR), wherein depending on various quantitative analysis methods, the buffer and fluorescent substances can be different, for example, DNA intercalation reagents (it can be EtBr, SYBR Green, etc.), turbidity reagents (it can be magnesium Pyrophosphate), fluorescent dyes (it can be FAM, Cy3 and Cy5), visible light detection or colorigenic reagent.

The heating source 20 is located at a bottom of the tube 10 or at a side thereof. The heating source 20 can be a thermally conductive sheet, and is for providing thermal conductive effectiveness with a heating source, or a heating source generally used in biochemical reactions, wherein the heating source 20 can be any manner which can achieve the same purpose of heating the tube 10.

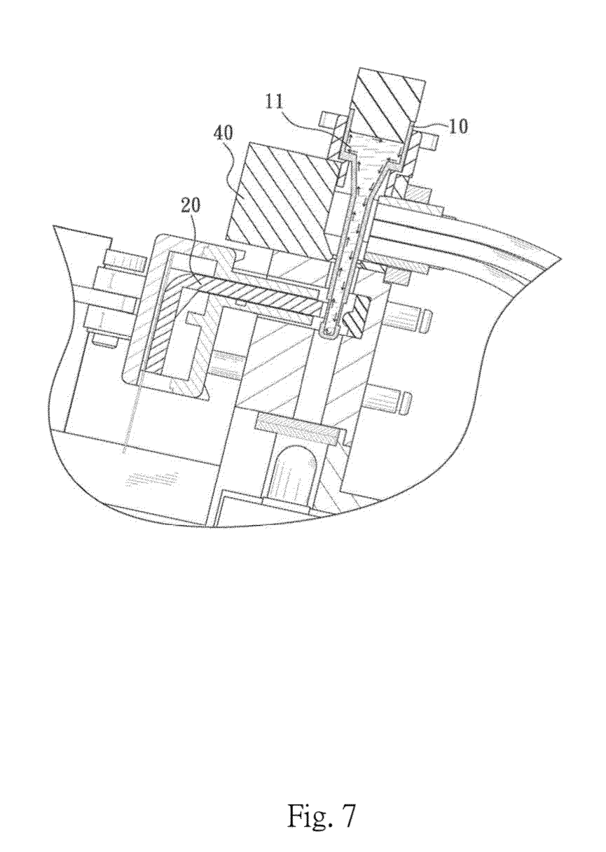

The heating source 20 can be controlled by a driving apparatus 21 to come in contact with the tube 10, thus the temperature of the buffer 11 (as shown in FIG. 7) in the tube 10 is the required temperature for nucleic acid amplification, wherein the bottom of the tube 10 is heated by the heating source 20 to increase the temperature of the buffer 11 to reach the required temperature for DNA denaturation, for which the optimal temperatures are 90 degrees Celsius to 99 degrees Celsius for current nucleic acid amplification. Furthermore, the temperature of the top of the tube 10 should be decreased below the required temperature for the primer annealing reaction. Therefore, the temperature difference of the buffer 11 between two ends of the tube 10 could be established to generate thermal convection.

The flow rate adjusting apparatus 30 is for controlling the flow direction of the thermal convection of the buffer 11 in the tube 10. The flow velocity and time of the buffer 11 are changed by controlling the flow direction of the buffer 11, hence the single thermal convection circulation period is equal to or longer than the single nucleic acid amplification period of the biochemical reaction to enhance the effectiveness of the nucleic acid amplification.

After understanding these main components and principles of the reaction apparatus, the operations and principles of the present disclosure are described in detail as follows.

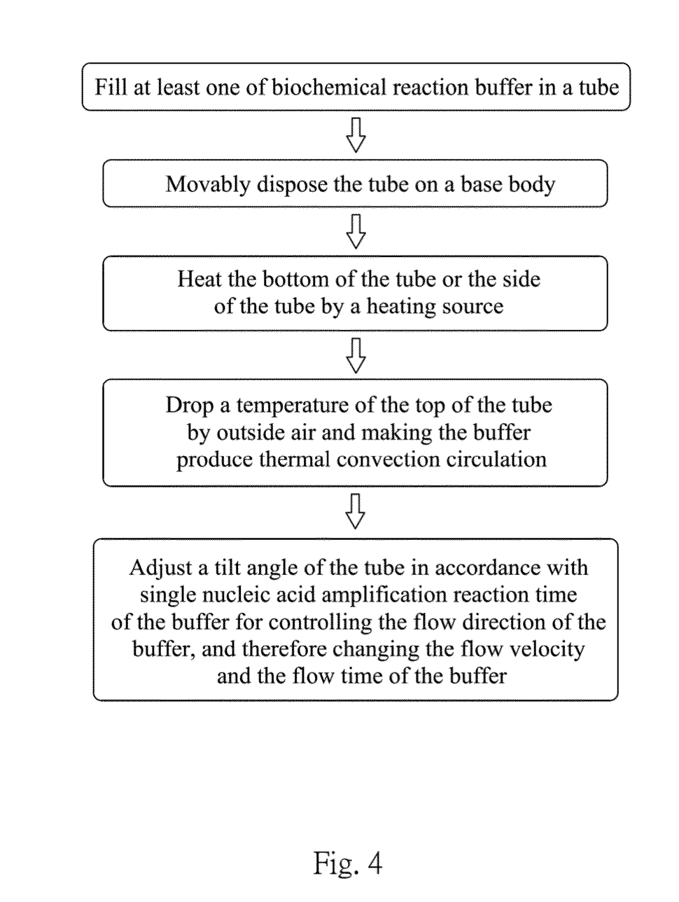

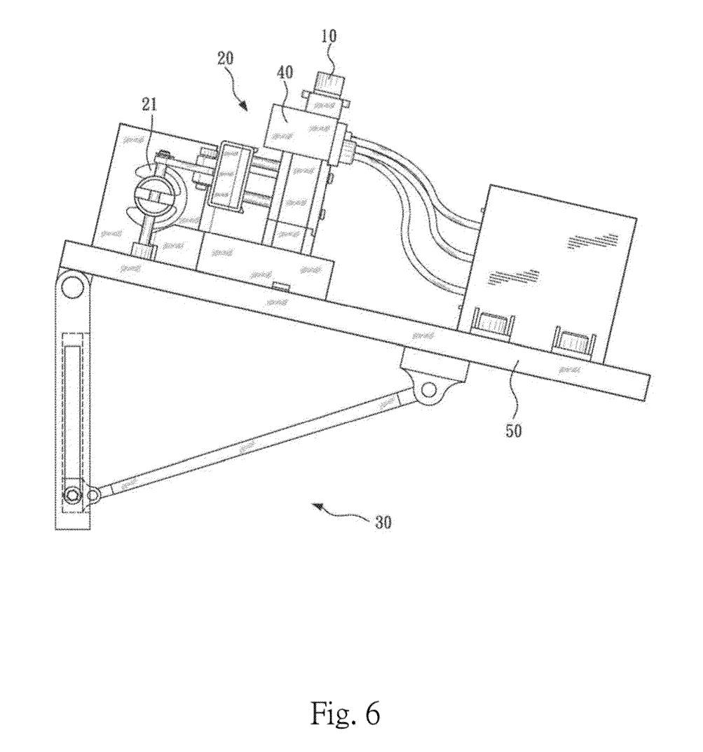

In FIG. 4 to FIG. 6, the protocol of the present disclosure includes the following steps:

a). at lea one buffer 11 of he biochemical reaction is filled in the tube 10;

b). the tube 10 which is movable is disposed on the base body 40;

c). the bottom of the tube 10 or the side of the tube 10 is heated by the heating source 20;

d). the temperature of the top of the tube 10 is decreased by outside air and a thermal convection circulation is generated in the buffer 11;

e). In accordance with the single nucleic acid amplification period needed in the buffer 11, t he tube 10 is adjusted at a tilt angle for controlling the flow direction of the buffer 11, and therefore the flow velocity and the flow time of the buffer 11 are changed.

FIG. 5 to FIG. 7 are schematic diagrams of the reaction apparatus in action according to one embodiment of the present disclosure. The base body 40 is disposed on a working bench 50, wherein the base body 40 is provided for disposing the tube 10 which is movable and the working bench 50 can be a flat plate. The flow rate adjusting apparatus 30 is connected to the working bench 50 for controlling the tilt angle of the working bench 50 and the base body 40. As the illustrations the flow rate adjusting apparatus 30 can drive the working bench 50 to adjust the angle through a connecting rod. The flow rate adjusting apparatus 30 also can be other structures which are used to adjust the angle of the working bench 50, for example, gear motor-mediated movement.

Through the control of the driving apparatus 21 the heating source 20 comes in contact with the tube 10 to heat the tube 10. When the temperature of the bottom of the tube 10 is increased, DNA in the buffer 11 will be denatured, when the buffer 11 flows to the top of the tube 10 to allow the buffer 11 to cool down, DNA in the buffer 11 will begin to replicate. Therefore, PCR amplification can be done in the buffer 11 during the circulation between the heating and cooling zones.

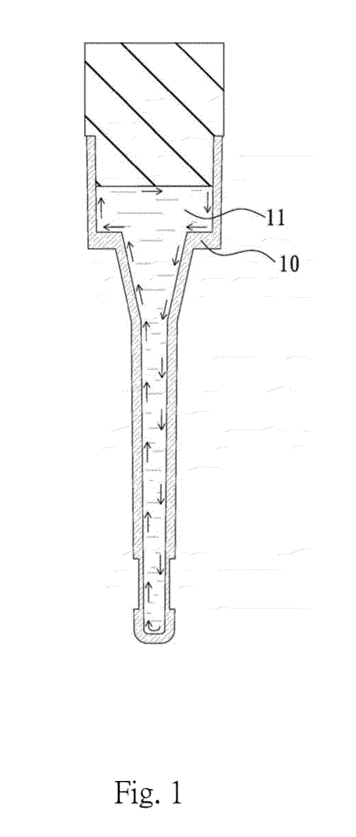

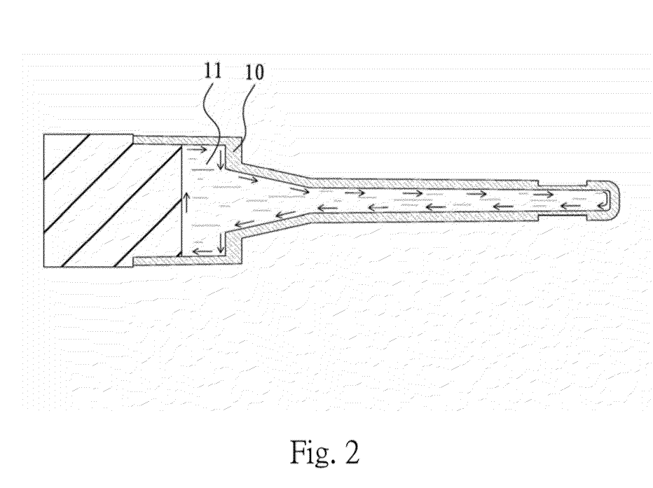

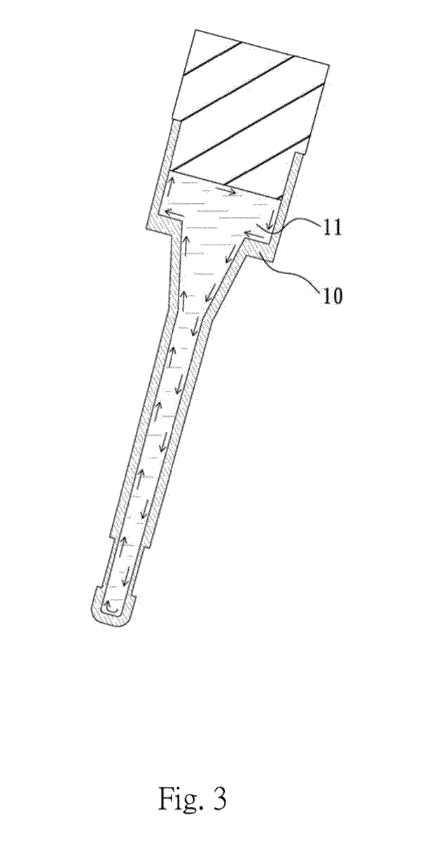

According to the above arrangement of configuration, in accordance with the required thermal convection velocity of the buffer 11, the base body 40 can drive the tube 10 to adjust the tilt angle to change the ratio of the change of the tube 10 in the vertical direction to the change of the tube 10 in the horizontal direction, thus the flow direction of the buffer 11 is changed. FIG. 1 to FIG. 3 are schematic diagrams of the tube 10 which is in a vertical, horizontal and inclined position, respectively. When the direction of the tube 10 is positioned closer to the vertical direction, the flow rate of the buffer 11 is higher. By contrast, when the direction of the tube 10 is positioned closer to the horizontal direction, the flow rate of the buffer 11 is lower. After experimental operation of the present disclosure, the angle between the arranged angle of the tube 10 and a vertical line over the ground can be greater than 0 and smaller than 45°, wherein the optimum angle is greater than 0 and smaller than 15° according to the optional thermal convection velocity of the buffer 11 which is required in current nucleic acid amplification.

The contact area between the wall of the tube 10 and the buffer flowing upward can be increased in thermal convection when the tube 10 is placed in the tilt angle. Thus, this can reduce the velocity and time of thermal convection circulation of the buffer 11, compared to the results in the tube 10 positioned in the vertical direction. Through this, the single thermal convection circulation period of the buffer 11 is equal to or longer than a single nucleic acid amplification period of the biochemical reaction. This could avoid the excessive circulation velocity in the single thermal convection, which may result in the single thermal convection circulation period of the buffer 11 to be shorter than the single nucleic, acid amplification period of DNA, in buffer influencing the results of nucleic acid amplification. Therefore, the present disclosure can effectively enhance the effectiveness of nucleic acid amplification.

In other embodiment of the present disclosure, in order to fix the tube 10 in the tilt angle, the flow rate adjusting apparatus 30 can be a bearing base (not shown) which is placed on the base body 40 and for holding the tube 10, and the bearing base (not shown) is capable of adjusting a tube angle on the base body 40, or an inclined plane (not shown) for holding the tube 10 can be included on the base body 40. In addition to all of the above methods, the tube 10 can be clamped by a hand-held tube holder, and the tube 10 can be tilted manually to achieve the sari purpose described above.

A method for steadying a thermal convection flow field in the PCR reaction solution during a thermal convective polymerase chain reaction (PCR)

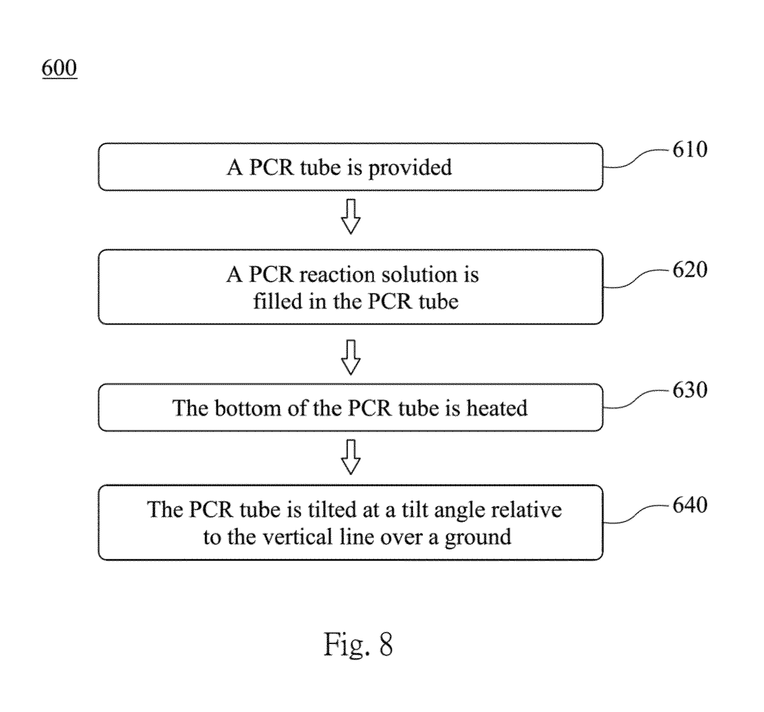

FIG. 8 is a flow diagram showing a method 600 for steadying a thermal convection flow field in the PCR reaction solution during a thermal convective PCR according to another embodiment of the present disclosure. In FIG. 8 the method 600 for steadying the thermal convection flow field in the PCR reaction solution during the thermal convective PCR includes a step 610, a step 620, a step 630 and a step 640.

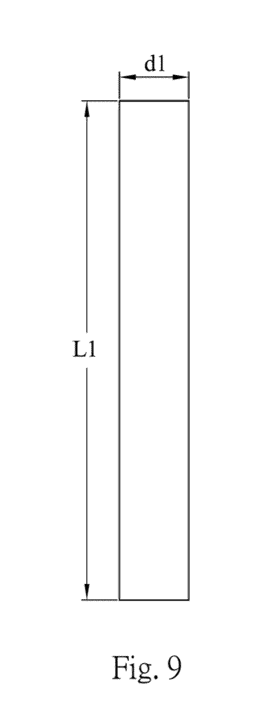

First, the step 610 is provided, wherein a PCR tube 700 is provided for containing a PCR reaction solution 710. FIG. 9 is a cross-sectional view of the PCR tube according to another embodiment of the present disclosure. A vertical length L1 between two ends of the PCR tube 700 can range from 5 mm to 45 mm. More particularly, the vertical length L1 between two ends of the PCR tube 700 can range from 25 mm to 45 mm. An inner diameter d1 of the PCR tube 700 can range from 0.3 mm to 5 mm. More particularly the inner diameter d1 of the PCR tube 700 can range from 0.5 mm to 5 mm.

Second, the step 620 is provided, wherein the PCR reaction solution 710 is filled in the PCR tube 700. The PCR reaction solution 710 includes DNA templates of a target nucleic acid sequence, DNA polymerase, dATP, dCTP, dGTP, dTTP, at least one primer pair for amplifying the target nucleic acid sequence and buffer, wherein the primer pair consists of a forward primer having a sequence complemented with 5′ end of the target nucleic acid sequence and a reverse primer having the sequence complemented with 3′ end of the target nucleic acid sequence. The PCR reaction solution 710 further includes the fluorescent substances. The fluorescent substances, for example SYBR Green I and SYTO-9, can bind non-specifically to DNA. The fluorescent substances, such as TaqMan probe and molecular beacon, which have the sequence complemented with the target nucleic acid sequence, a fluorescent group in the 5′ end, and a corresponding quencher in the 3′ end, can also bind specifically to DNA.

Then; the step 630 is provided, wherein the bottom of the PCR tube 700 is heated to the temperature required for the denaturation of the PCR by the heating source. The temperature required for the denaturation of the PCR usually ranges from 90° C. to 98° C. A thermal convection is induced by the difference of the temperature at the top of the PCR reaction solution 710 and the temperature at the bottom of the PCR reaction solution 710. The heating source is a simple heating device with temperature control members, such as a dry bath heater, a water bath heater or oil bath heater,

Finally, the step 640 is provided, wherein the PCR tube 700 is tilted at a tilt angle relative to the vertical line over a ground for increasing the probability of a single-loop flow in the thermal convection flow field in the PCR reaction solution 710 during the thermal convective PCR, wherein the tilt angle ranges from 1 to 89. Accordingly, the different temperature regions are formed in a bottom section, a middle section and a top section of the PCR reaction solution 710, whereby the denaturation, the annealing reaction and the extension reaction are occurred sequentially in different temperature regions of the PCR reaction solution 710. The denaturation, the annealing reaction and the extension reaction also occur repeatedly by the thermal convection circulation, so that the target nucleic acid sequence can be amplified to the required amount to complete the PCR. Furthermore, the performance of the thermal convective PCR is consistent among different PCR tubes 700 under the same reaction condition.

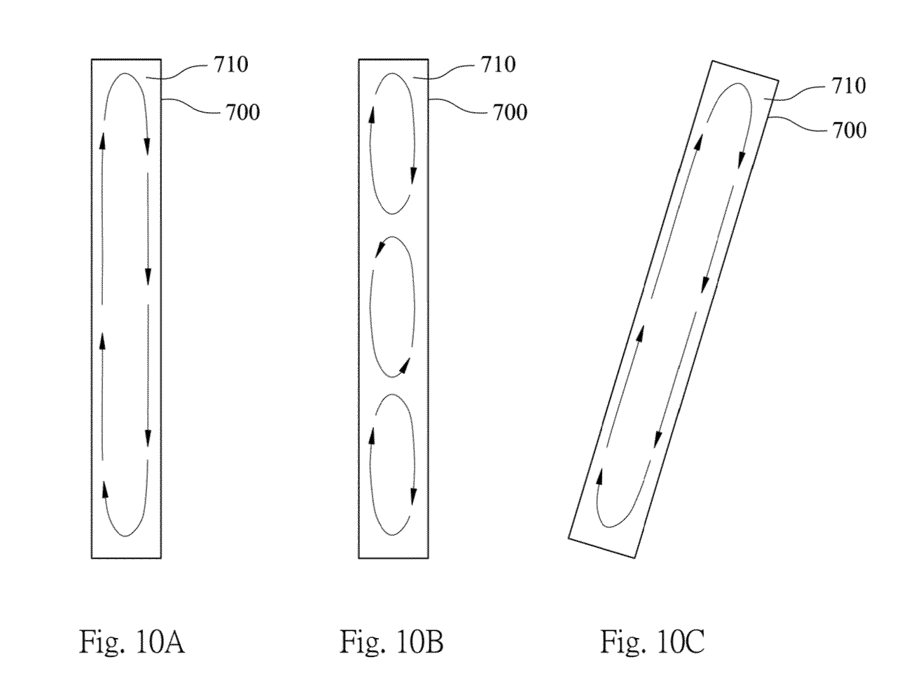

FIG. 10A is a schematic diagram of an ideal thermal convection flow field in the PCR tube which is in a vertical position during the thermal convective PCR. FIG. 10B is a schematic diagram of an actual thermal convection flow field in the PCR tube which is in the vertical position during the thermal convective PCR. FIG. 10C is a schematic diagram of the thermal convection flow field in I he PCR tube which is in an inclined position during the thermal convective PCR according to another embodiment of the present disclosure. In FIG. 10A, the ideal thermal convection flow field is the single-loop flow. However, when PCR tube 700 is placed in the vertical position, the flow of the PCR reaction solution 710 in the PCR tube 700 randomly starts from the left side of the PCR reaction solution 710 or from the right side of the PCR reaction solution 710. The temperature gradient at the bottom of the PCR reaction solution 710 is larger than that at the top of the PCR reaction solution 710, thus the driven force of the liquid flow at the bottom of the PCR reaction solution 710 is greater than the driven force of the liquid flow at the top of the PCR reaction solution 710. Therefore, the convection flow field in the thermal convective PCR can have not only the single-loop flow but also a two-loop flow or a multi-loop flow (FIG. 10B). Accordingly, the PCR performance consistency between runs is poor because the PCR reaction solution 710 can't flow throw through all regions of different temperatures available in the PCR reaction solution 710 and only circulates in different limited regions of temperatures among different PCR tubes 700 containing the same PCR reaction solution 710. By contrast, when PCR tube 700 is placed in the inclined position (FIG. 10C). The hotter PCR reaction solution 710 prefers to stay on the up side of the PCR reaction solution 710, and the colder PCR reaction solution 710 prefers to stay on the lower side of the PCR reaction solution 710. Consequently, the single-loop flow could be established in the flow field in the PCR reaction solution 710 during the thermal convective PCR in the PCR tube 700. The PCR solution will flow throw all regions of different temperatures available in the PCR reaction solution 710 to enable the performance of the thermal convective PCR to be consistent among different PCR tubes 700 containing the same PCR reaction solution 710.

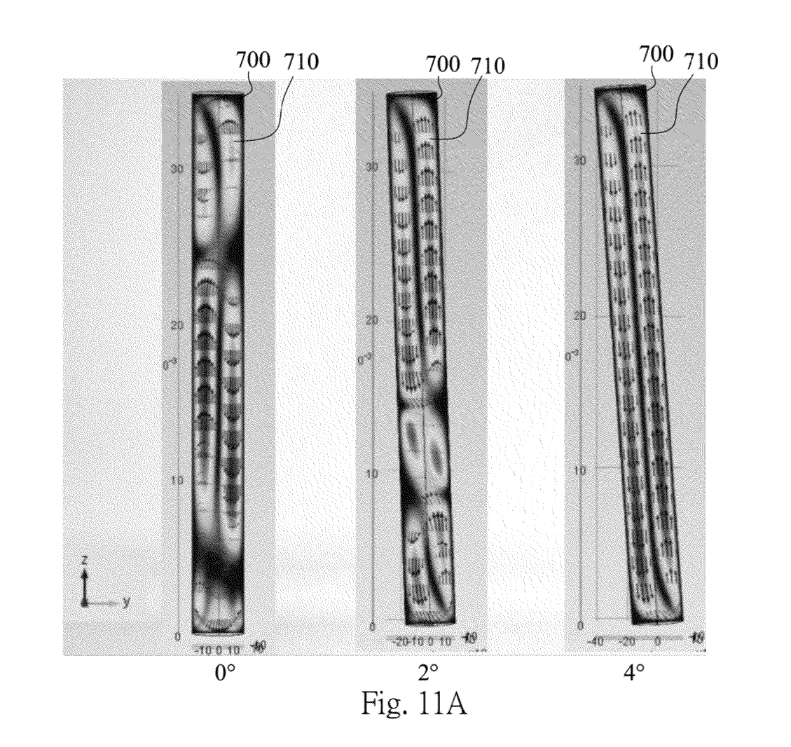

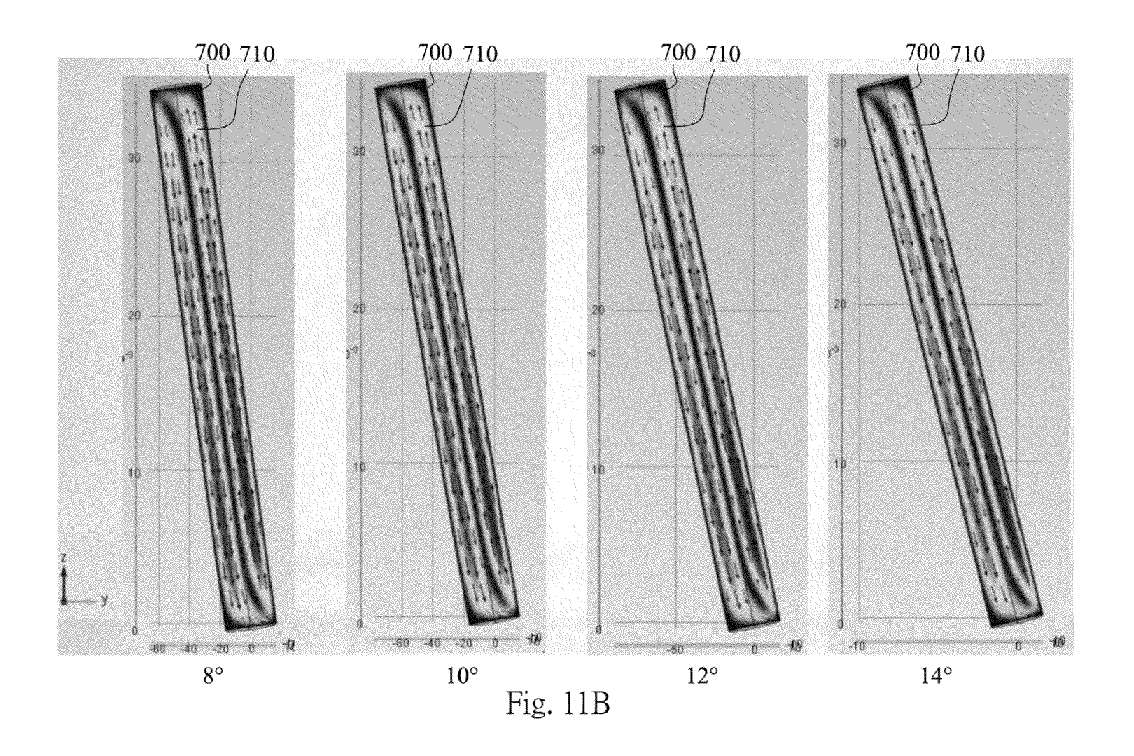

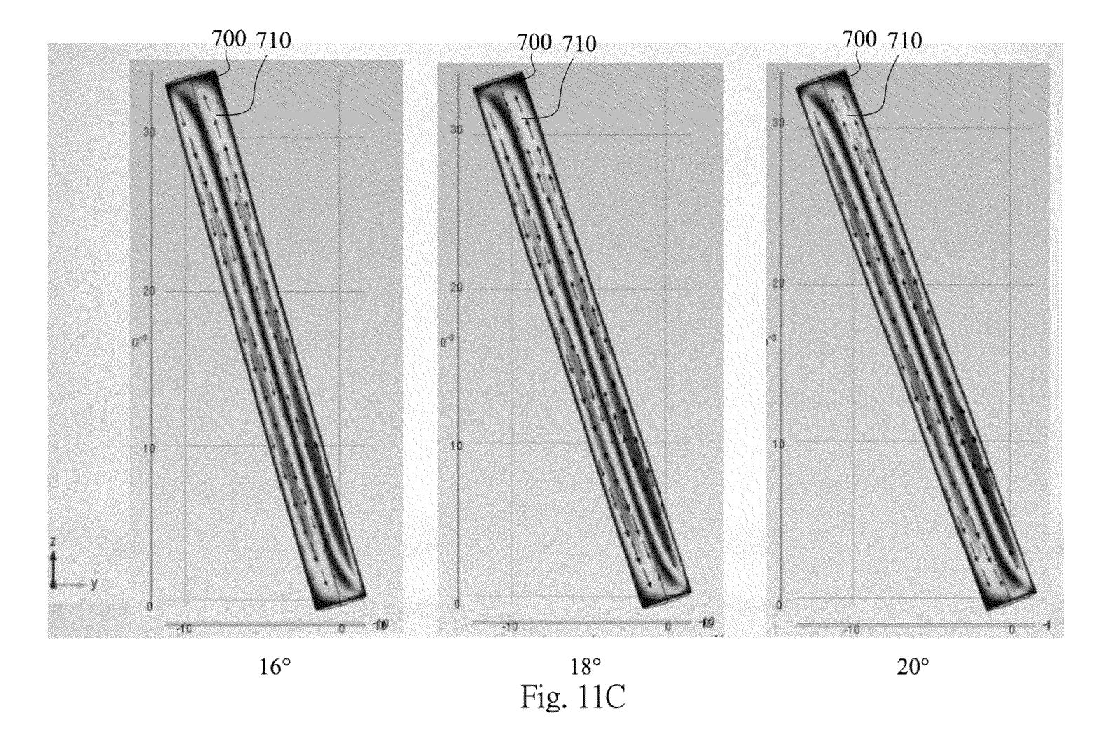

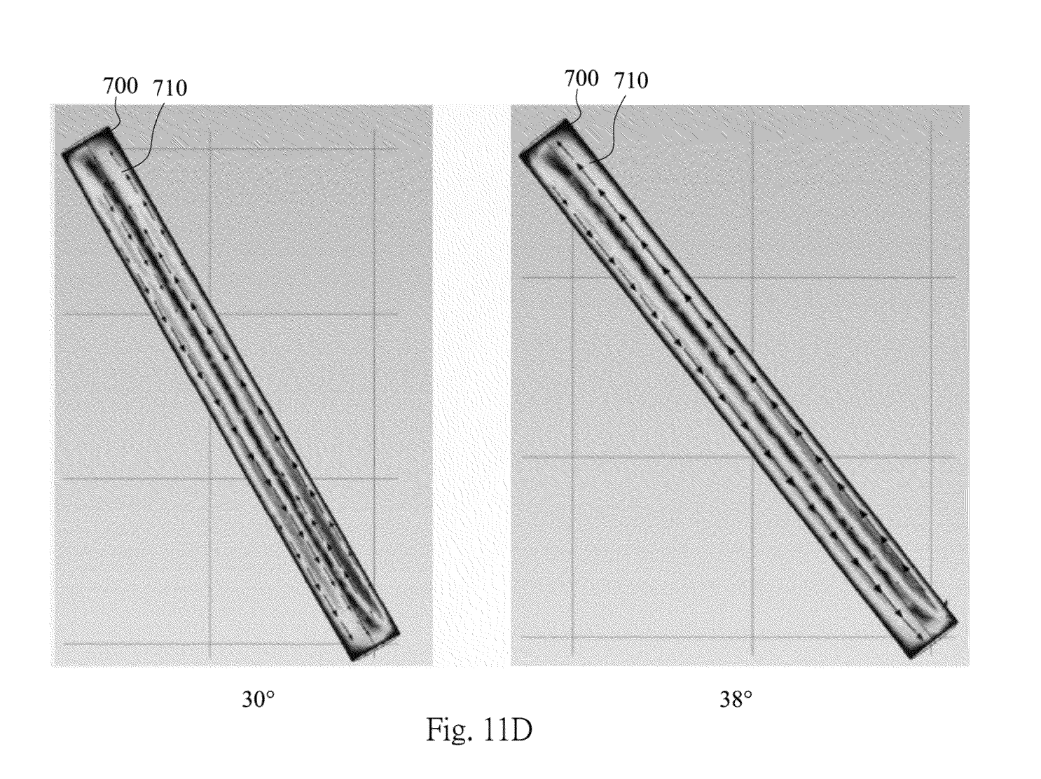

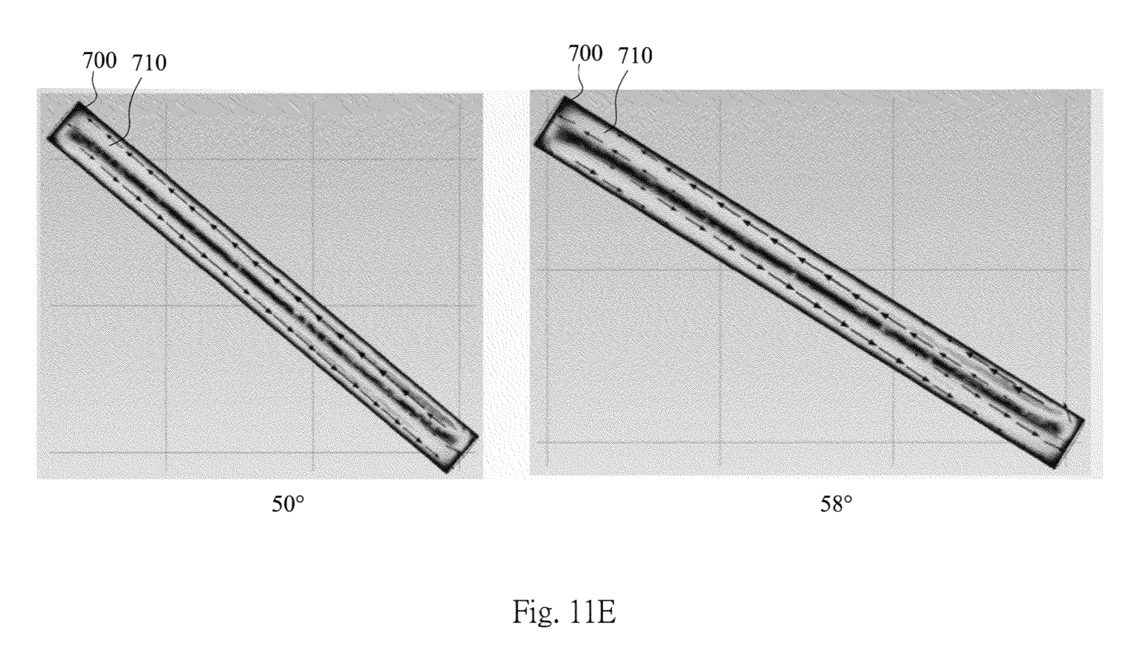

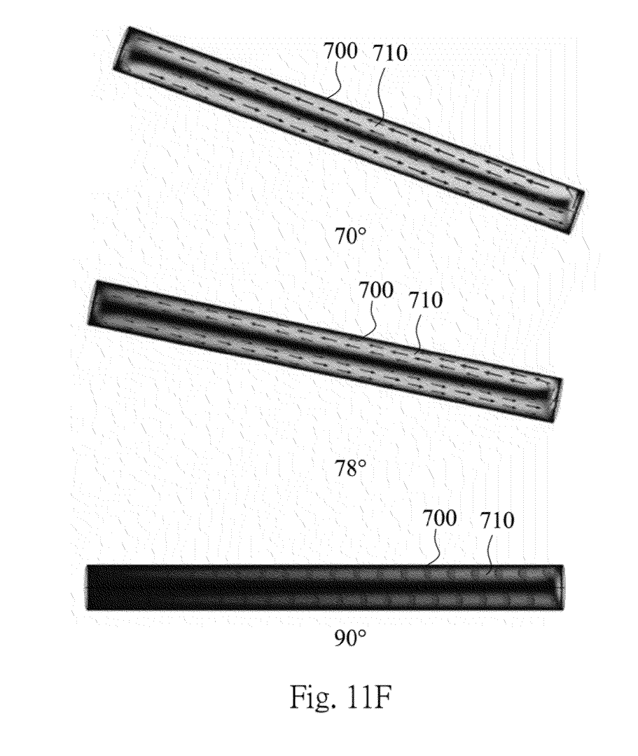

Referring to FIGS. 11A to 11 and Table 1, FIGS. 11 to 11F are diagrams of computer-simulation fluid dynamics in the thermal convection flow field in the PCR tube 700 at different tilt angles according to one example of the present disclosure. In this example, the vertical length between two ends of the PCR tube 700 is 35 mm, the inner diameter of the PCR tube 700 is 1.65 mm, and the test solution is water. Table I shows the flow field type and the maximum flow rate (Vmax) in the PCR tube 700 at different tilt angles during the thermal convective PCR.

TABLE 1

| Tilt angle | Flow field type | Vmax (m/s) |
|---|---|---|
| 0° | two-loop flow | 8.22 × 10⁻³ |
| 2° | multi-loop flow | 7.9 × 10⁻³ |
| 4° | single-loop flow | 8.38 × 10⁻³ |
| 8° | single-loop flow | 8.84 × 10⁻³ |
| 10° | single-loop flow | 8.89 × 10⁻³ |
| 12° | single-loop flow | 9.19 × 10⁻³ |
| 14° | single-loop flow | 9.32 × 10⁻³ |
| 16° | single-loop flow | 9.43 × 10⁻³ |
| 18° | single-loop flow | 9.48 × 10⁻³ |
| 20° | single-loop flow | 9.32 × 10⁻³ |
| 30° | single-loop flow | 8.98 × 10⁻³ |
| 38° | single-loop flow | 8.40 × 10⁻³ |
| 50° | single-loop flow | 7.73 × 10⁻³ |
| 58° | single-loop flow | 7.29 × 10⁻³ |
| 70° | single-loop flow | 6.08 × 10⁻³ |
| 78° | single-loop flow | 5.08 × 10⁻³ |
| 90° | single-loop flow | 3.63 × 10⁻³ |

Under the test condition that the vertical length between two ends of the PCR tube 700 is 35 mm, the inner diameter of the PCR tube 700 is 1.65 mm, and the test solution is water, the thermal convection flow field in the PCR reaction solution 710 in the PCR tube 700, which is placed in the vertical position (tilt angle of 0°), is the two-loop flow in the top region and the middle region of the PCR tube 700 and random and chaotic natural convection in the bottom region of the PCR tube 700. When the PCR tube 700 is tilted at 2°, the thermal convection flow field in the PCR reaction solution 710 in the PCR tube 700 includes a three-loop flow with the loops locating separately in the top region the middle, region and the bottom region of the PCR tube 700. When the PCR tube 700 is tilted at or greater than 4′, the thermal convection flow field in the PCR reaction solution 710 in the PCR tube 700 includes the single-loop flow, facilitating stable fluid circulation from the bottom of the PCR tube 700 to the top of the PCR tube 700. In addition, when the PCR tube 700 is tilted at 8° to 90°, the thermal convection flow field in the PCR reaction solution 710 in the PCR tube 700 has a stable single-loop flow. The results indicate that tilting the PCR tube 700 at the tilt angle relative to the vertical line over the ground can help eliminating the two-loop flow or the multi-loop flow and establishing the stable single-loop flow under the same test condition (the same vertical length of the PCR tube 700, the same inner diameter of the PCR tube 700 and the same test solution). Furthermore, when the PCR tube 700 is tilted at 2° to 18°, the maximum flow rate is increased with the increased tilt angle. When the PCR tube 700 is tilted at 20° to 90°, the maximum flow rate is decreased with the increased tilt angle.

For highlighting the method for steadying the thermal convection flow field in the PCR reaction solution during the thermal convective PCR of the present disclosure can steady the thermal convection flow field into the single-loop flow by tilting the PCR tube 700 at the tilt angle relative to the vertical line over the ground. The above example demonstrates test conditions that, at the small tilt angles ranged from 0° to 4°, the two-loop flow or multi-loop flow is formed in the thermal convection flow field. However, in other examples, the thermal convection flow field also can be driven into the single-loop flow at these small tilt angles by adjusting the vertical length of the PCR tube 700, the inner diameter of the PCR tube 700 or a viscosity of the PCR reaction solution 710. The viscosity of the PCR reaction solution 710 can be increased by adding non-reactive organic substances or non-reactive inorganic substances, such as glycerol, NP-40, Tween 20, EDTA, DMSO, formamide, betaine or gelatin.

To demonstrate the application of the method for steadying the thermal convection flow field in the PCR reaction solution during the thermal convective PCR of the present disclosure can enhance the performance consistency of the thermal convective PCR in different PCR tubes 700 containing the same PCR reaction solution 710. Two thermal convective PCR devices (device I and device H) are used to perform the thermal convective PCR in this example. The vertical length between two ends of the PCR tube 700 used in this example is 35 mm, and the inner diameter of the PCR tube 700 used in this example is 1.65 mm. The PCR reaction solution 710 includes DNA templates of the target nucleic acid sequence, the forward primer having a sequence complemented with 5 end of the target nucleic acid sequence, the reverse primer having the sequence complemented with 3′ end of the target nucleic acid sequence, fluorescent probe, dATP, dCTP, dGTP, dTTP, DNA polymerase and buffer.

Sterilize deionized water is added into the PCR reaction solution 710 for adjusting the total volume of the PCR reaction solution 710 to 50 μl. The PCR tube 700 is heated near the bottom for fifty minutes at 95° C. to obtain the reaction results. There are 8 PCR tubes 700 to perform the thermal convective PCR in each thermal convective PCR device.

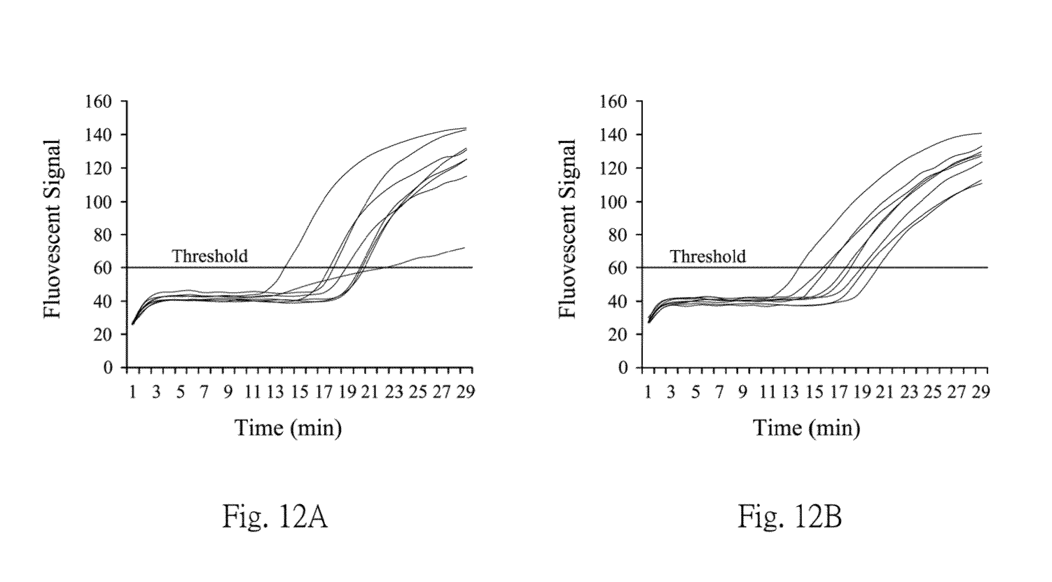

Referring to FIGS. 12A to 12B and Table 2, FIG. 12A is the diagram of fluorescent signals generated in the thermal convective PCR by using device wherein the PCR tube 700 is in the vertical position during the thermal convective PCR. FIG. 12B is the diagram of fluorescent signals generated in the thermal convective PCR by using device II, wherein the PCR tube 700 is in the vertical position during the thermal convective PCR. There are 8 PCR tubes 700 to perform the thermal convective PCR in the device I and the device II. Table 2 shows the average time of fluorescent signal reached to the threshold in the 8 PCR tubes 700 and the standard deviation.

TABLE 2

| | Device I | Device II |
|---|---|---|
| Average time for fluorescent signal | 18.1 minutes | 16.9 minutes |
| to reach the threshold | | |
| Standard deviation | 2.5 | 2.3 |

The results show that the average time f©r fluorescent signal to reach the threshold varies among the 8 PCR tubes 700, which are placed in the vertical position. The standard deviation of the time for the fluorescent signal to reach the threshold among 8 PCR tubes 700 in the device I and device II is 2.5 and 2.3 respectively. The results indicate that the performance of the thermal convective PCR is not consistent among different PCR tubes 700 containing the same PCR reaction solution 710 under these conditions.

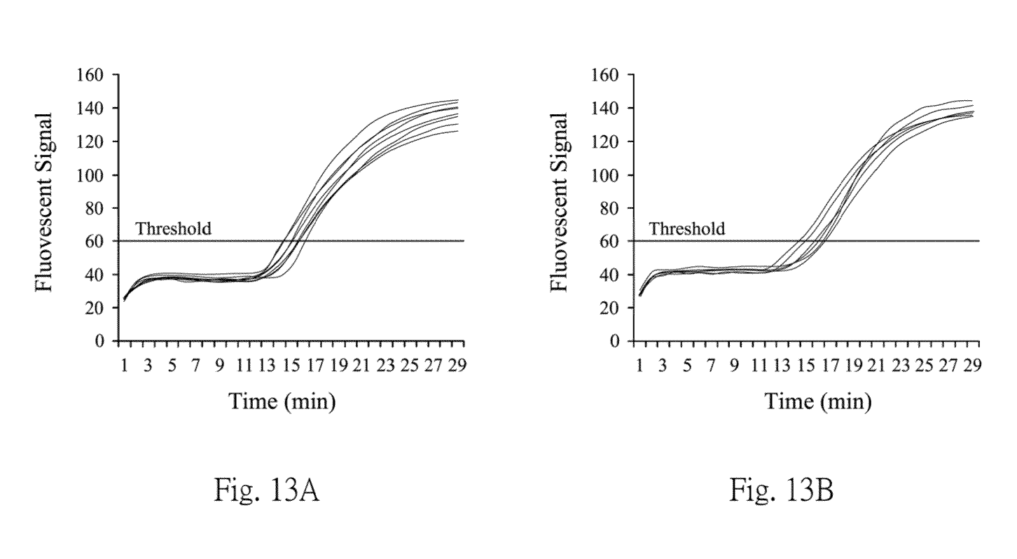

Referring to FIGS. 13A to 13B and Table 3, FIG. 13A is the diagram of fluorescent signals generated in the thermal convective PCR by using device I, wherein the PCR tube 700 is tilted at 20 during the thermal convective PCR. FIG. 13B is the diagram of fluorescent signals generated in the thermal convective PCR by using device II wherein the PCR tube 700 is tilted at 20° during the thermal convective PCR. There are also 8 PCR tubes 700 to perform the thermal convective PCR in the device I and the device II. Table 3 shows the average time for the fluorescent signal to reach to the threshold in the 8 PCR tubes 700 and the standard deviation.

TABLE 3

| | Device I | Device II |
|---|---|---|
| Average time for fluorescent signal | 14.8 minutes | 15.3 minutes |
| to reach the threshold | | |
| Standard deviation | 0.8 | 1.0 |

The results show that the average time for the fluorescent signal to reach the threshold in the 8 PCR tubes 700, which are tilted at 20° during the thermal convective PCR, are similar under the same test condition in the device I and device II. The standard deviation of the time for the fluorescent signal to reach the threshold among the 8 PCR tubes 700 in the device I and device II is decreased to 0.8 and 1.0 respectively. The results indicate that the method for steadying the thermal convection flow field in the PCR reaction solution during the thermal convective PCR of the present disclosure can enhance the consistency of the performance of the thermal convective PCR in different PCR tubes 700 containing the same PCR reaction solution 710.

To sum up, the present disclosure provides the method for steadying the thermal convection flow field in the PCR reaction solution during the thermal convective PCR. The aforementioned method can increase the probability of establishing the single-loop flow in the thermal convection flow field in the PCR reaction solution during the thermal convective PCR by tilting the PCR tube at the tilt angle relative to the vertical line over the ground. For creating the optimal thermal convective PCR condition, the vertical length of the PCR tube and t he inner diameter of the PCR tube can be adjusted in order to control the thermal convection time. The stable thermal circulation can result in that the temperature gradients of the PCR reaction solution are similar in different tubes, enabling conditions for the denaturation, the annealing reaction and the extension reaction to be consistent in different tubes, and improving efficiency of the thermal convective PCR. The performance consistency of the thermal convective PCR in different PCR tubes containing the same PCR reaction solution is enhanced, and the target nucleic acid sequence can be amplified at similar efficiency. Therefore, the aforementioned method can mitigate the problem that the thermal convective PCR performance is not consistent in different PCR tubes containing the same PCR reaction solution when the PCR tubes are placed in the vertical position, wherein the inconsistent thermal convective PCR performance is due to the difference between the flow rates at the top of the PCR reaction solution and the flow rates at the bottom of the PCR reaction solution, leading to not only the single-loop flow but also the two-loop flow or the multi-loop flow in the thermal convective flow field.

Although the present disclosure has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present disclosure without departing from the scope or spirit of the invention. In view of the foregoing, it is intended that the present disclosure cover modifications and variations of this disclosure provided they fall within the scope of the following claims.

What is claimed is:

1. A method for steadying a thermal convection flow field in a PCR reaction solution during a thermal convective polymerase chain reaction, comprising:
providing a PCR tube;
filling the PCR reaction solution in the PCR tube;
heating a bottom of the PCR tube so that a thermal convection is induced; and
tilting the PCR tube at a tilt angle relative to a vertical line over a ground for causing a single-loop flow in the thermal convection flow field in the PCR reaction solution;
whereby a denaturation, an annealing reaction and an extension reaction occur sequentially and repeatedly in different temperature regions of the PCR reaction solution.

2. The method for steadying the thermal convection flow field in the PCR reaction solution during the thermal convective polymerase chain reaction of claim 1, wherein the tilt angle ranges from 1 to 89.

3. The method for steadying the thermal convection flow field in the PCR reaction solution during the thermal convective polymerase chain reaction of claim 1, wherein a vertical length between two ends of the PCR tube ranges from 5 mm to 45 mm.

4. The method for steadying the thermal convection flow field in the PCR reaction solution during the thermal convective polymerase chain reaction of claim 1, wherein the vertical length between two ends of the PCR tube ranges from 25 mm to 45 mm.

5. The method for steadying the thermal convection flow field in the PCR reaction solution during the thermal convective polymerase chain reaction of claim 1, wherein an inner diameter of the PCR tube ranges from 0.3 mm to 5 mm.

6. The method for steadying the thermal convection flow field in the PCR reaction solution during the thermal convective polymerase chain reaction of claim 1, wherein the inner diameter of the PCR tube ranges from 0.5 mm to 5 mm.
